(19) United States
(12) Patent Application Publication
(10) Pub. No.: US 2024/0351014 A1
(43) Pub. Date: Oct. 24, 2024

(54) CO2-MEDIATED OXIDATIVE DEHYDROGENATION OF PROPANE OVER MODIFIED VOx/Al2O3 CATALYST COMPOSITION

(71) Applicant: KING FAHD UNIVERSITY OF PETROLEUM AND MINERALS, Dhahran (SA)

(72) Inventors: Mohammad Mozahar HOSSAIN, Dhahran (SA); Yahya GAMBO, Dhahran (SA); Mohammed Saleh BA-SHAMMAKH, Dhahran (SA); Sagir ADAMU, Dhahran (SA)

(73) Assignee: KING FAHD UNIVERSITY OF PETROLEUM AND MINERALS, Dhahran (SA)

(21) Appl. No.: 18/302,045

(22) Filed: Apr. 18, 2023

(51) Int. Cl. B01J 37/08; B01J 6/00; B01J 21/02; B01J 23/22; B01J 35/00; B01J 35/02; B01J 35/10; B01J 37/00; B01J 37/04; C07C 5/333

(52) U.S. Cl. CPC B01J 37/086; B01J 6/001; B01J 21/02; B01J 23/22; B01J 35/19; B01J 35/40; B01J 35/613; B01J 35/633; B01J 35/635; B01J 35/647; B01J 37/0036; B01J 37/04; C07C 5/3332

(57) ABSTRACT

A method of making an active catalyst composition includes mixing an alumina support with first catalyst precursor particles and grinding thereby at least partially embedding the first catalyst precursor particles onto surfaces of the alumina support to form a first composite precursor; mixing the first composite precursor and a first solvent to form a first mixture; grinding the first mixture and drying at a temperature of 100 to 150° C.; calcining the first mixture after the drying at a first temperature of at least 200° C. and a second temperature of at least 550° C. thereby allowing the first catalyst precursor particles embedded onto the surfaces of the alumina support to decompose in situ to generate first catalyst particles embedded onto the surfaces of the alumina support and form a first catalyst; and mixing the first catalyst with a second catalyst to form the active catalyst composition.

### BACKGROUND

### Technical Field

The present disclosure is directed to a catalyst, particularly a VOₓ/Al₂O₃ catalyst composition for CO₂-mediated oxidative dehydrogenation of propane, and a method of making the catalyst and a method of using the catalyst for dehydrogenation of propane.

### Description of Related Art

The “background” description provided herein is to present the context of the disclosure generally. Work of the presently named inventors, to the extent it is described in this background section, as well as aspects of the description that may not otherwise qualify as prior art at the time of filing, are neither expressly nor impliedly admitted as prior art against the present invention.

Boron-based catalysts are used for the oxidative dehydrogenation of light alkanes to olefins, mainly due to their ability to limit cracking and overoxidation reaction pathways. These catalysts include but are not limited to boron nitrides, boron oxides, boron phosphates, doped boron materials. The design and tuning strategies employed for boron-based catalysts include morphology modulation to yield layered superstructures with an ability to stabilize intermediate species and target olefin products. The active sites of these catalysts can be constructed as edge-adsorbed oxygen species, defective trigonal boron species, edge-hydroxylated species, or carbonyl groups and oxygen-terminated armchair edges in carbon-doped boron catalysts.

The catalytic function of boron-based catalysts can be modulated by embedding them in suitable materials such as graphitic carbon. The boron-based catalysts can also be supported on nanodiamonds, engineering and confining defects such as non-metal oxygen vacancies within boron nitride. Additional methods include tuning the crystallinity of boron species and using plasma treatment to modify the local environment of the boron species.

On the other hand, vanadium-based catalysts are among the most studied catalysts for the CO₂—ODH process and other selective redox processes, largely due to their tuneable surface properties and promising performances. However, the challenge has remained to construct lattice oxygens that can achieve an optimal balance between activity and selectivity. This is because highly active lattice oxygens tend to show low selectivity to olefins owing to their electron-deficient nature, while electron-rich (nucleophilic) lattice oxygens are usually highly selective but limited by their low catalytic activity. Thus, there is a critical need for a catalyst composition that can effectively leverage the benefits of both classes of catalysts and overcome their respective limitations.

Accordingly, one objective of the present disclosure is to develop a catalyst composition that simultaneously harnesses the advantages of the two classes of catalysts and circumvents their individual limitations by environmentally friendly protocols. A further objective of the present disclosure is to describe a method for producing propylene via oxidative dehydrogenation of propane.

### SUMMARY

In an exemplary embodiment, a method of making an active catalyst composition containing a first catalyst and a second catalyst is described. The method includes mixing an alumina support having an average particle size of 5 to 100 micrometers (μm) with first catalyst precursor particles and grinding thereby at least partially embedding the first catalyst precursor particles onto surfaces of the alumina support to form a first composite precursor. In some embodiments, a weight ratio of the alumina support to the first catalyst precursor particles is in a range of 100:1 to 5:1. The method further includes mixing the first composite precursor and a first solvent to form a first mixture. The method also includes grinding the first mixture and drying at a temperature of 100 to 150° C. The method also includes calcining the first mixture after the drying at a first temperature of at least 200° C. and a second temperature of at least 550° C. thereby allowing the first catalyst precursor particles embedded onto the surfaces of the alumina support to decompose in situ to generate first catalyst particles embedded onto the surfaces of the alumina support and form the first catalyst. In some embodiments, the first catalyst particles have an average particle size in a range of 20 to 200 nanometers (nm). In some embodiments, the first catalyst particles comprise vanadium oxide (VOₓ) particles, and boron oxide (B₂O₃) particles, in which 0<x<3. Additionally, the method includes mixing the first catalyst with the second catalyst to form the active catalyst composition.

In some embodiments, the alumina support is at least one selected from the group consisting of alpha-alumina, delta-alumina, theta-alumina, and gamma-alumina.

In some embodiments, the first catalyst precursor particles comprise a vanadium compound selected from the group consisting of vanadium acetylacetate, vanadium acetylacetonate, ammonium vanadate, vanadyl oxalate, vanadium pentoxide, vanadium monoethanolamine, vanadium chloride, vanadium trichloride oxide, vanadyl sulfate, vanadium antimonate, antimony vanadate, vanadium oxyacetylacetonate, vanadium oxyacetate, vanadium oxyhalide, and vanadium oxytriisopropoxide.

In some embodiments, the first catalyst precursor particles comprise a boron compound selected from the group consisting of boric acid, boron nitride, borax, boron halide, and borane halide.

In some embodiments, the vanadium oxide (VOₓ) comprises vanadium monoxide (VO), vanadium trioxide (V₂O₃), vanadium dioxide (VO₂), and vanadium pentoxide (V₂O₅).

In some embodiments, the method of making the active catalyst composition further includes forming the second catalyst. The method includes mixing the first catalyst with second catalyst precursor particles and grinding thereby at least partially embedding the second catalyst precursor particles onto the surfaces of the alumina support to form a second composite precursor. A weight ratio of the first catalyst to the second catalyst precursor particles is in a range of 100:1 to 9:1. The method further includes mixing the second composite precursor and a second solvent to form a second mixture; grinding the second mixture and drying at a temperature of 100 to 150° C.; and calcining the second mixture after the drying at a first temperature of at least 200° C. and a second temperature of at least 550° C. thereby allowing the second catalyst precursor particles to decompose in situ to generate second catalyst particles embedded onto the surfaces of the alumina support and form the second catalyst. The second catalyst particles have an average particle size in a range of 20 to 200 nm. In addition, the second catalyst particles comprise vanadium oxide particles, and boron oxide particles.

In some embodiments, the second solvent is at least one selected from the group consisting of a ketone solvent, an ester solvent, an alcohol solvent, an amide solvent, and an ether solvent.

In some embodiments, the second composite precursor is present in the second mixture at a concentration of 40 to 80 wt. % based on a total weight of the second mixture.

In some embodiments, the active catalyst composition is at least one selected from the group consisting of an alumina supported vanadium oxide (VOₓ), an alumina supported boron oxide (B₂O₃), and an alumina supported VOₓ/B₂O₃; and wherein 0<x<3.

In some embodiments, the active catalyst composition has a layered mesoporous structure.

In some embodiments, the active catalyst composition has a specific surface area in a range of 50 to 100 square meters per gram (m²/g).

In some embodiments, the active catalyst composition has a cumulative specific pore volume in a range of 0.2 to 0.7 cubic centimeters per gram (cm³/g).

In some embodiments, the active catalyst composition has an average pore diameter of 50 to 400 angstroms (Å).

In some embodiments, the active catalyst composition has a temperature-programmed desorption of ammonia (NH₃-TPD) of 0.01 to 0.5 millimoles per gram (mmol/g).

In some embodiments, the active catalyst composition has a hydrogen temperature-programmed reduction (H₂-TPR) of 0.01 to 0.7 mmol/g.

In an exemplary embodiment, a method for producing propylene via oxidative dehydrogenation (ODH) of propane is described. The method includes introducing a feed gas stream containing CO₂ and propane into a reactor containing the active catalyst composition; passing the feed gas stream through the reactor in the presence of the active catalyst composition at a temperature of 300 to 900° C. to convert at least a portion of the propane to propylene and produce a propylene-containing gas stream leaving the reactor; and separating the propylene from the propylene-containing gas stream.

In some embodiments, a volume ratio of CO₂ to propane in the feed gas stream is in a range of 1:10 to 10:1.

In some embodiments, the propylene-containing gas stream further comprises methane, ethane, ethylene, propane, carbon monoxide, carbon dioxide, hydrocarbon containing C₄-C₅, and aromatics.

In some embodiments, the method has a propane conversion of up to 80% based on an initial weight of the propane in the feed gas stream.

In some embodiments, the method has a propylene yield of up to 50% based on the propane conversion according to equation Y₃=(X_{C}_{₃}_{H}_{₈}*S₃)×100%. In some embodiments, X_{C}_{₃}_{H}_{₈} denotes the propane conversion to propylene. In some embodiments, S₃ denotes the propylene selectivity.

The foregoing general description of the illustrative present disclosure and the following The detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is a schematic illustration depicting a catalyst model VOₓ/Al₂O₃(A1V), B₂O₃/Al₂O₃(A1B), and VOₓ—B₂O₃(x)/Al₂O₃(AlV03B and A1V07B), according to certain embodiments;

FIG. 1B is a schematic illustration depicting a catalyst model B₂O₃/Al₂O₃ (A1B), according to certain embodiments;

FIG. 1C is a schematic illustration depicting a catalyst model VOₓ—B₂O₃(x)/Al₂O₃(AlV03B and A1V07B), according to certain embodiments;

FIG. 2A is a flow chart depicting a method of making an active catalyst composition, according to certain embodiments;

FIG. 2B is a flow chart depicting a method for producing propylene via oxidative dehydrogenation (ODH) of propane, according to certain embodiments;

FIG. 3A is a plot depicting N₂ adsorption-desorption isotherm and non-local density functional theory (NLDFT) pore size distribution of the A1V catalyst, according to certain embodiments;

FIG. 3B is a plot depicting the N₂ adsorption-desorption isotherms and NLDFT pore size distribution of the AlB catalyst, according to certain embodiments;

FIG. 3C is a plot depicting the N₂ adsorption-desorption isotherms and NLDFT pore size distribution of the AlV03B catalyst, according to certain embodiments;

FIG. 3D is a plot depicting the N₂ adsorption-desorption isotherms and NLDFT pore size distribution of the AlV07B catalyst, according to certain embodiments;

FIG. 4A is a full-range Fourier Transform Infrared (FTIR) spectra stacked with individual y-axis offsets of the various catalysts, according to certain embodiments;

FIG. 4B depicts a zero-offset stacked FTIR spectra highlighting peaks appearing above 1000 cm⁻¹ of the various catalysts, according to certain embodiments;

FIG. 5A shows a diffuse reflectance UV-Vis spectra (UV-vis DRS) of the various catalysts at an absorbance versus wavelength (200-800 nm), according to certain embodiments;

FIG. 5B is a plot of (αhν)² versus incident photoenergy for band gap evaluation of the various catalysts, according to certain embodiments;

FIG. 6 is a Raman spectrum of the various catalysts (fresh), according to certain embodiments;

FIG. 7A shows a H₂ temperature programmed reduction (H₂-TPR) profile for the various catalysts, according to certain embodiments;

FIG. 7B shows a deconvoluted profile for the AlV catalyst depicting various peaks, according to certain embodiments;

FIG. 8A shows a NH₃ temperature programmed desorption (NH₃-TPD) plot of the various catalysts according to certain embodiments;

FIG. 8B shows a deconvoluted thermal conductivity detector (TCD) signal for the AlV catalyst depicting the various peaks, according to certain embodiments;

FIG. 9A shows propane conversion for the various catalysts during CO₂-mediated oxidative dehydrogenation (CO₂—ODH)) of propane, according to certain embodiments;

FIG. 9B depicts CO₂ conversion for the various catalysts during the CO₂—ODH of propane, according to certain embodiments;

FIG. 9C depicts the propylene yield with the various catalysts during the CO₂—ODH of propane, according to certain embodiments;

FIG. 9D depicts the CO selectivity with the various catalysts during the CO₂—ODH of propane, according to certain embodiments;

FIG. 10A shows the catalytic stability of the AlVO7B catalyst in terms of propane conversion and CO₂ conversion, according to certain embodiments;

FIG. 10B shows the product selectivity of the AlVO7B catalyst towards propylene, ethylene, methane, ethane, and CO over 12 h time on stream (TOS), according to certain embodiments;

FIG. 11 is a thermal gravimetric analysis (TGA) plot of spent AlV07B catalyst with TOS, according to certain embodiments; and

FIG. 12 is a schematic illustration depicting the fabrication of suitable active site diversity to promote selectivity in the CO₂—ODH process over VOx-Box/γ-Al₂O₃ catalyst, according to certain embodiments.

### DETAILED DESCRIPTION

In the drawings, reference numerals designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

As used herein, the term ‘calcination’ refers to the thermal treatment of a solid chemical compound whereby the compound is raised to a high temperature without melting under a restricted supply of ambient oxygen, generally for the purpose of removing impurities or volatile substances and to incur thermal decomposition.

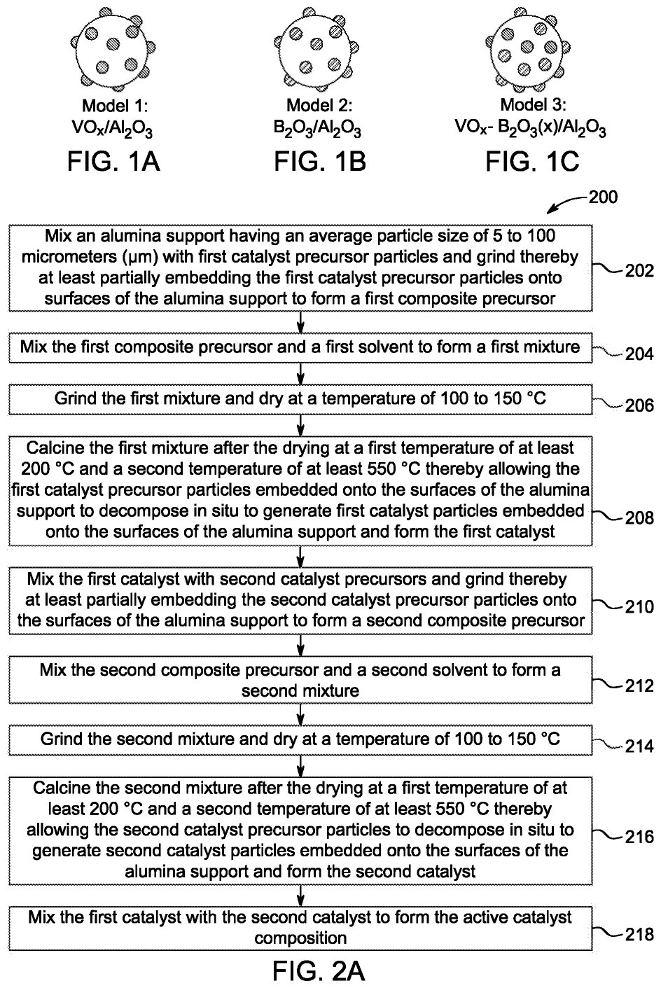

Aspects of the present disclosure are directed to the design of efficient heterogeneous catalysts for CO₂-oxidative dehydrogenation (ODH) of light alkanes. The CO₂—ODH process holds prospects in enabling the direct valorization of anthropogenic CO₂ and largely underutilized shale and natural gas resources. Controlled integration of both VOx and Box species was performed to develop efficient catalysts with tuned active site diversity. The solvent- and template-free synthetic design strategy involved precisely incorporating the active components into a T-Al₂O₃ support via physical grinding, e.g., in a mortar, to yield the integrated catalysts, and a two-phase calcination protocol. The design models of the various catalysts investigated, namely, VOₓ/Al₂O₃ (labeled A1V), are depicted in FIG. 1A, B₂O₃/Al₂O₃ (labelled A1B) is depicted in FIG. 1B, and VOₓ—B₂O₃(x)/Al₂O₃ is depicted in FIG. 1C.

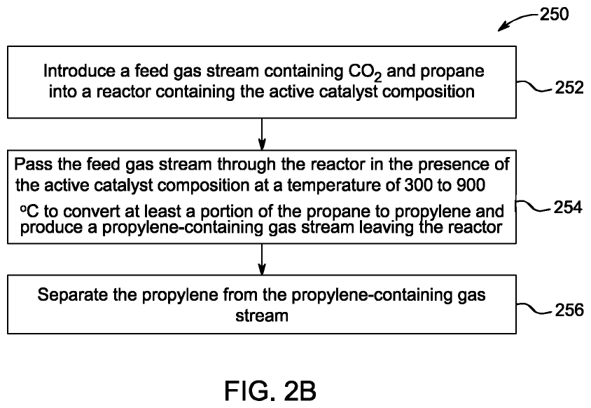

Referring to FIG. 2A, a schematic flow diagram of the method 200 of making an active catalyst composition containing a first catalyst and a second catalyst is illustrated. The order in which the method 200 is described is not intended to be construed as a limitation, and any number of the described method steps may be combined in any order to implement the method 200.

Additionally, individual steps may be removed or skipped from the method 200 without departing from the spirit and scope of the present disclosure.

At step 202, the method 200 includes mixing an alumina support having an average particle size of 5 to 100 micrometers (μm) with first catalyst precursor particles and grinding thereby at least partially embedding the first catalyst precursor particles onto surfaces of the alumina support to form a first composite precursor. In some embodiments, the alumina support has an average particle size of 5 to 100 μm, preferably 10 to 80 μm, preferably 15 to 60 μm, or even more preferably 20 to 40 μm. Other ranges are also possible. In some embodiments, the alumina support is selected from alpha-alumina, delta-alumina, theta-alumina, and gamma-alumina group. In a preferred embodiment, the alumina support is gamma-alumina. In some embodiments, the gamma-alumina has an excellent surface area owing to the small particle size, which results in the high activity of the surface for catalyst support. Optionally, other support materials, such as silica, silica gel, alumina, titania, silica/alumina, calcium metasilicate, pyrogenic silica, high purity silica, zirconia, carbon, zeolites, and mixtures thereof, can be used as support materials as well (albeit with a few variations, as may be obvious to a person skilled in the art). In some embodiments, the support may be impregnated with promoters. Suitable examples of the promoters include one or more transition metals, one or more transition metal-containing compounds, alkali metals, alkali-metal-containing compounds, or combinations thereof.

The alumina support may be procured commercially. In an exemplary process of making the alumina support, the alumina support is immersed in an aqueous solution (preferably water) or any other liquid to form a paste-like substance. The paste is further heated to a rate of 10° C./min, preferably about 5° C./min, and more preferably about 3° C./min up to 180° C., preferably up to 160° C., preferably up to 140° C., or even more preferably up to 120° C. Other ranges are also possible. Heating to such temperature results in the evaporation of water molecules from the alumina support; it may also alter its pore structure. The support is further dried to a temperature range of 100-140° C., preferably to about 120° C., to obtain the alumina support. The alumina support thus obtained can be used to prepare the active catalyst composition. In a preferred embodiment, the alumina support is amorphous. In certain embodiments, the alumina support may be prepared by other methods conventionally known in the art. The alumina support may be porous or non-porous. In some embodiments, the gamma-alumina has a closely packed hexagonal structure in which aluminum and oxygen atoms are arranged in a regular pattern. In some further embodiments, the aluminum atoms are located at the centers of octahedra, and the oxygen atoms occupy tetrahedral and octahedral sites in a way that creates a distorted hexagonal close-packed arrangement. In some preferred embodiments, the alumina support has a specific surface area of up to 200 μm²/g, more preferably up to 100 μm²/g. In some further preferred embodiments, the alumina support has a pore volume in a range of 0.1 to 1.0 cm³/g, preferably 0.2 to 0.8 cm³/g, or even more preferably 0.3 to 0.6 cm³/g. Other ranges are also possible.

The alumina support, preferably gamma-alumina, was mixed with first catalyst precursor particles. The first catalyst precursor particles include a vanadium compound and a boron compound. Suitable examples of the vanadium compound include vanadium acetylacetate, vanadium acetylacetonate, ammonium vanadate, vanadyl oxalate, vanadium pentoxide, vanadium monoethanolamine, vanadium chloride, vanadium trichloride oxide, vanadyl sulfate, vanadium antimonate, antimony vanadate, vanadium oxyacetylacetonate, vanadium oxyacetate, vanadium oxyhalide, and vanadium oxytriisopropoxide. Suitable examples of the boron compound include boric acid, boron nitride, borax, boron halide, and borane halide. In a preferred embodiment, the vanadium compound is vanadyl acetyl acetate precursor. In a preferred embodiment, the boron compound is boric acid. In some embodiments, the first catalytic precursor is a boron compound, preferably, boric acid. The first catalyst precursor particles are mixed with the alumina support and ground for 5-30 minutes, preferably 5-10 minutes, to obtain the composite. The weight-by-weight ratio of the alumina support to the first catalyst precursor particles is in a range of 100:1 to 5:1, preferably 80:1 to 10:1, preferably 60:1 to 15:1, or even more preferably 40:1 to 20:1. Other ranges are also possible.

In some embodiments, the first catalyst precursor particles are at least partially embedded onto surfaces of the alumina support. In some embodiments, at least 30% by number of the first catalyst precursor particles are embedded onto the surfaces of the alumina support, preferably at least 50%, preferably at least 70%, or even more preferably at least 90%. In some further embodiments, no more than 95% by number of the first catalyst precursor particles are embedded onto the surfaces of the alumina support, preferably no more than 75%, preferably no more than 55%, or even more preferably no more than 35%. Other ranges are also possible.

At step 204, the method 200 includes mixing the first composite precursor and a first solvent to form a first mixture. The first solvent is at least one selected from the group consisting of a ketone solvent, an ester solvent, an alcohol solvent, an amide solvent, and an ether solvent. In some embodiments, the first solvent is an alcohol—such as ethanol, isopropyl alcohol, etc. In a preferred embodiment, the first solvent is ethanol. In some embodiments, the first composite precursor is present in the first mixture at a concentration of 1 to 20 wt. % based on the total weight of the first mixture, preferably 2 to 15 wt. %, preferably 3 to 10 wt. %, preferably 4 to 6 wt. %, or even more preferably about 5 wt. % based on the total weight of the first mixture. Other ranges are also possible.

At step 206, the method 200 includes grinding the first mixture and drying at a temperature of 100 to 150° C., preferably 110 to 140° C., or even more preferably 120 to 130° C. In some embodiments, the first mixture was ground for 1 to 60 minutes, preferably 2-30 minutes, preferably 5-10 minutes, or even more preferably about 5 minutes, till the first mixture was dispersed entirely in the composite. The first mixture was further dried to a temperature of 70 to 100° C., preferably 80-100° C., preferably 95° C. to evaporate the solvent molecules from the first mixture. The drying process was carried out in an oven for 1-5 hours, preferably 2-4 hours, or more preferably for 2 hours. Other ranges are also possible.

At step 208, the method 200 includes calcining the first mixture after the drying at a first temperature of at least 200° C. and a second temperature of at least 550° C. thereby allowing the first catalyst precursor particles embedded onto the surfaces of the alumina support to decompose in situ to generate first catalyst particles embedded onto the surfaces of the alumina support and form the first catalyst. The calcination process may involve one or more steps. In some embodiments, the calcination process involves two steps—for example, in the first step, the calcination was carried out by heating the first mixture to a temperature range of 200-400° C., preferably to about 300° C. at a heating rate of 1-20° C./min, preferably 3-15° C./min, preferably 5-10° C./min, more preferably at about 5° C./min. This process was carried out for about 1-5 hours, preferably 2-3 hours, and more preferably for about 2 hours. In the second step, the first mixture was further heated to a temperature range of 550° C.-800° C., preferably 600-750° C., preferably 625-675° C., and more preferably to about 675° C. at a heating rate of 1-20° C./min, preferably 3-15° C./min, preferably 5-10° C./min, more preferably at about 10° C./min. This step was carried out for 2-12 hours, preferably 5-10 hours, and more preferably to about 6 hours. Other ranges are also possible. The calcination may be performed by any conventional method or apparatus known to a person skilled in the art, for example, but not limited to, using a muffle furnace, electric furnace, tube furnace, box furnace, crucible furnace, microwave furnace, vacuum furnace, rotary kiln, or fluidized bed furnace. The two-step calcination process decomposes the first catalyst precursor to form the first catalyst particles, which are deposited on the alumina support to form the first catalyst. In some embodiments, the first catalyst particles have an average particle size in a range of 20 to 200 nm, preferably 75 to 175 nm, preferably 50 to 150 nm, preferably 75 to 125 nm, or even more preferably about 100 nm. Other ranges are also possible.

The first catalyst particles deposited on the alumina support may be vanadium oxide (VOₓ) particles, where 0<x<3. The vanadium in the VOₓ particles may exist in various oxidation states, such as +2, +3, +4, and +5. The VOₓ particles include particles of vanadium monoxide (VO), vanadium trioxide (V₂O₃), vanadium dioxide (VO₂), and/or vanadium pentoxide (V₂O₅). In some embodiments, the first catalyst particles deposited on the alumina support may be boron oxide particles. In a preferred embodiment, the first catalyst particles deposited on the alumina support include a combination of vanadium and boron oxide particles.

In some embodiments, the first catalyst precursor particles embedded onto the surfaces of the alumina support decomposes in situ during the calcining to generate first catalyst particles embedded onto the surfaces of the alumina support and form the first catalyst. In some embodiments, at least 50% by number of the first catalyst precursor particles are decomposed to generate first catalyst particles. In some embodiments, at least 30% by number of the first catalyst particles are at embedded onto the surfaces of the alumina support, preferably at least 50%, preferably at least 70%, or even more preferably at least 90%. In some further embodiments, no more than 95% by number of the first catalyst particles are at embedded onto the surfaces of the alumina support, preferably no more than 75%, preferably no more than 55%, or even more preferably no more than 35%. Other ranges are also possible.

The first catalyst particles may be of any shape, such as spherical, cubical, ellipsoid, and the like. In a preferred embodiment, the first catalyst particles are spherical. In some embodiments, the first catalyst particles are uniformly distributed throughout the first catalyst.

At step 210, the method 200 includes mixing the first catalyst with second catalyst precursor particles and grinding thereby at least partially embedding the second catalyst precursor particles onto the surfaces of the alumina support to form a second composite precursor. The second catalyst precursor particles include the vanadium compound and/or the boron compound. Suitable examples of the vanadium compound include vanadium acetylacetate, vanadium acetylacetonate, ammonium vanadate, vanadyl oxalate, vanadium pentoxide, vanadium monoethanolamine, vanadium chloride, vanadium trichloride oxide, vanadyl sulfate, vanadium antimonate, antimony vanadate, vanadium oxyacetylacetonate, vanadium oxyacetate, vanadium oxyhalide, and vanadium oxytriisopropoxide. Suitable examples of the boron compound include boric acid, boron nitride, borax, boron halide, and borane halide. In a preferred embodiment, the vanadium compound is a vanadyl acetyl acetate precursor. In a preferred embodiment, the boron compound is boric acid. In some embodiments, the second catalyst precursor particles are the vanadium compound, preferably vanadyl acetyl acetate. The weight ratio of the first catalyst to the second catalyst precursor particles is in a range of 100:1 to 9:1, preferably 80:1 to 20:1, preferably 60:1 to 40:1, or even more preferably about 50:1. Other ranges are also possible.

At step 212, the method 200 includes mixing the second composite precursor and a second solvent to form a second mixture. The second solvent is at least one selected from a ketone solvent, an ester solvent, an alcohol solvent, an amide solvent, and an ether solvent. In some embodiments, the second solvent is alcohol—such as ethanol, isopropyl alcohol, etc. In a preferred embodiment, the second solvent is ethanol. In some embodiments, the second composite precursor is present in the second mixture at a concentration of 40 to 80 wt. % based on the total weight of the second mixture, preferably 50 to 70 wt. %, or even more preferably about 60 wt. % based on the total weight of the second mixture.

At step 214, the method 200 includes grinding the second mixture and drying at a temperature of 100 to 150° C. The mixture was ground for 2-20 minutes, preferably 5-10 minutes, more preferably for 5 minutes, till the mixture was dispersed entirely in the first catalyst. The second mixture was further dried to a temperature of 100 to 150° C., preferably 110-140° C., preferably 120° C. to evaporate the solvent molecules from the mixture. The drying process was carried out in an oven for 1-5 hours, preferably 2-4 hours, or more preferably for 2 hours. Other ranges are also possible.

At step 216, the method 200 includes calcining the second mixture after the drying at a first temperature of at least 200° C. and a second temperature of at least 550° C. thereby allowing the second catalyst precursor particles to decompose in situ to generate second catalyst particles embedded onto the surfaces of the alumina support and form the second catalyst. The calcination process involves one or more steps. In some embodiments, the calcination process involves two steps—for example, in the first step, the calcination was carried out by heating the composite to a temperature range of 200-400° C., preferably to about 300° C. at a heating rate of 1-20° C./min, preferably 3-15° C./min, preferably 5-10° C./min, more preferably at about 5° C./min. This process was carried out for about 1-5 hours, preferably 2-3 hours, and more preferably for about 2 hours. In the second step, the mixture was further heated to a temperature range of 550° C.-800° C., preferably 600-750° C., preferably 625-675° C., and more preferably to about 675° C. at a heating rate of 1-20° C./min, preferably 3-15° C./min, preferably 5-10° C./min, more preferably at about 10° C./min. This step was carried out for 2-12 hours, preferably 5-10 hours, and more preferably to about 6 hours. The two-step calcination process decomposes the second catalyst precursor to form the second catalyst particles, with an average particle size in a range of 20 to 200 nm.

The second catalyst precursor particles are decomposed and deposited onto the surface of the first catalyst, to form the second catalyst. In some embodiments, at least 50% by number of the second catalyst precursor particles are decomposed to generate the second catalyst particles. In some embodiments, the second catalyst precursor particles are at least partially embedded onto surfaces of the first catalyst. In some embodiments, at least 30% by number of the second catalyst precursor particles are embedded onto the surfaces of the first catalyst, preferably at least 50%, preferably at least 70%, or even more preferably at least 90%. In some further embodiments, no more than 95% by number of the second catalyst precursor particles are embedded onto the surfaces of the first catalyst, preferably no more than 75%, preferably no more than 55%, or even more preferably no more than 35%. Other ranges are also possible.

In some embodiments, the second catalyst particles deposited on the surface of the first catalyst composition are vanadium oxide (VOₓ) particles, where 0<x<3. The vanadium in the VOₓ particles may exist in various oxidation states, such as +2, +3, +4, and +5. The VOₓ particles include particles of vanadium monoxide (VO), vanadium trioxide (V₂O₃), vanadium dioxide (VO₂), and/or vanadium pentoxide (V₂O₅). In some embodiments, the second catalyst particles are boron oxide particles. In some embodiments, the second catalyst particles include particles of vanadium oxide (VOₓ) and boron oxide (B₂O₃).

At step 218, the method 200 includes mixing the first catalyst with the second catalyst to form the active catalyst composition. The first catalyst and a second catalyst together form the active catalyst composition. The active catalyst composition is at least one selected from the group consisting of an alumina-supported vanadium oxide (VOx), an alumina-supported boron oxide (B₂O₃), and an alumina-supported VOx/B₂O₃; and where 0<x<3. The active catalyst composition has a layered mesoporous structure with a cumulative specific pore volume in a range of 0.1 to 0.8 cm³/g, preferably 0.2 to 0.7 cm³/g, preferably 0.3 to 0.6 cm³/g, or even more preferably 0.4 to 0.5 cm³/g, and an average pore diameter of 50 to 400 Å, preferably 100 to 300 Å, or even more preferably 150 to 250 Å. In some embodiments, the specific surface area of the active catalyst composition is in the range of 50 to 100 μm²/g, preferably 100 to 150 μm²/g, or even more preferably about 125 μm²/g. Other ranges are also possible.

The catalysts were characterized via N₂-adsorption analysis, Fourier Transform Infrared (FTIR) spectroscopy, Raman spectroscopy, diffuse reflectance UV-Vis spectra (UV-vis DRS), CO₂ temperature programmed desorption (CO₂-TPD), and H₂ temperature programmed reduction (H₂-TPR) analysis.

As used herein, the term “temperature program reduction using H₂,” or “H2-TPR”, generally refers to a technique used to study the reducibility of a solid material, such as an active catalyst composition, by measuring the consumption of a reducing gas, such as hydrogen, as a function of temperature. In some embodiments, the active catalyst composition is first heated in an oxidizing gas, such as air or oxygen, to remove any adsorbed species and to convert the active catalyst composition to an oxide. In some further embodiments, the active catalyst composition is then cooled down and exposed to a stream of hydrogen gas, while the temperature is gradually increased. As the temperature increases, the hydrogen reacts with the oxidized active catalyst composition, causing a reduction of the material. In some preferred embodiments, this reduction reaction may be exothermic, and the heat generated by the reaction is monitored as a function of temperature.

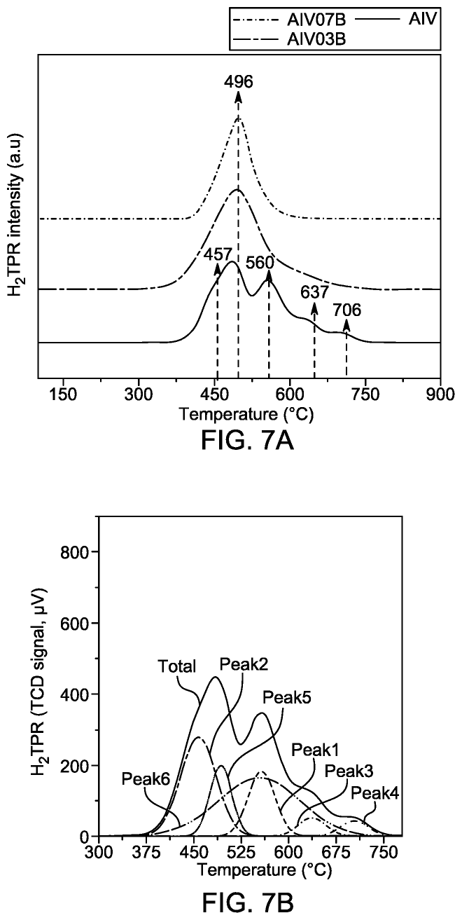

Referring to FIG. 7A, hydrogen-temperature programmed reduction (H₂-TPR) plots of the active catalyst compositions. In some embodiments, the H₂-TPR was conducted on a Micromeritics AutoChem-II 2920 unit equipped with a TCD detector. The active catalyst was placed in a quartz calcined at a temperature of 200 to 400° C., preferably 225 to 375° C., preferably 250 to 350° C., preferably 275 to 325° C., or even more preferably about 300° C. under an argon flow for at least 60 minutes, at least 120 minutes, at least 180 minutes. In some further embodiments, the active catalyst was cooled to a temperature of no more than 70° C., preferably no more than 60° C., or even more preferably no more than 50° C. In some preferred embodiments, a gas flow contains hydrogen (H₂) and argon (Ar) in a volumetric ratio of H₂ to Ar ranging from 1:20 to 1:1, preferably 1:15 to 1:5, or even more preferably about 1:10 was introduced to flow over the active catalyst composition at a flow rate of 10 to 100 cubic centimeters per minutes (cm³/min), preferably 30 to 70 cm³/min, or even more preferably about 50 cm³/min. In some preferred embodiments, the temperature of the analyzer containing the active catalyst was increased at ramping rate of 5 to 20° C. per minute until the temperature reaches about 900° C. Other ranges are also possible. In some embodiments, H₂-TPR of the active catalyst composition are about 0.01 to 0.7 mmol/g, preferably about 0.05 to 0.6 mmol/g, preferably 0.1 to 0.5 mmol/g, preferably 0.2 to 0.4 mmol/g, or even more preferably about 0.3 mmol/g, as depicted in FIG. 7A. Other ranges are also possible.

As used herein, the term “N₂ adsorption/desorption method” generally refers to a technique used to measure the specific surface area of a solid material, such as a catalyst or a porous material. In some embodiments, the active catalyst composition is exposed to a stream of nitrogen gas at low temperature and pressure. The nitrogen gas is adsorbed onto the surface of the active catalyst composition, filling the pores and creating a monolayer of adsorbed nitrogen. In some further embodiments, the amount of nitrogen adsorbed at a given pressure is measured using a gas adsorption instrument, such as a BET instrument. In some preferred embodiments, the BET analysis is performed on a BELCAT II Chemisorption analyzer according to the software manual, manufactured by Bell Japan. In some more preferred embodiments, the nitrogen gas is gradually removed from the active catalyst composition, causing the desorption of the adsorbed nitrogen. The amount of nitrogen desorbed at a given pressure is also measured using the gas adsorption instrument. By analyzing the amount of nitrogen adsorbed and desorbed, the specific surface area of the active catalyst can be calculated using the BET (Brunauer-Emmett-Teller) and Barrett, Joyner and Halenda (BJH) equation.

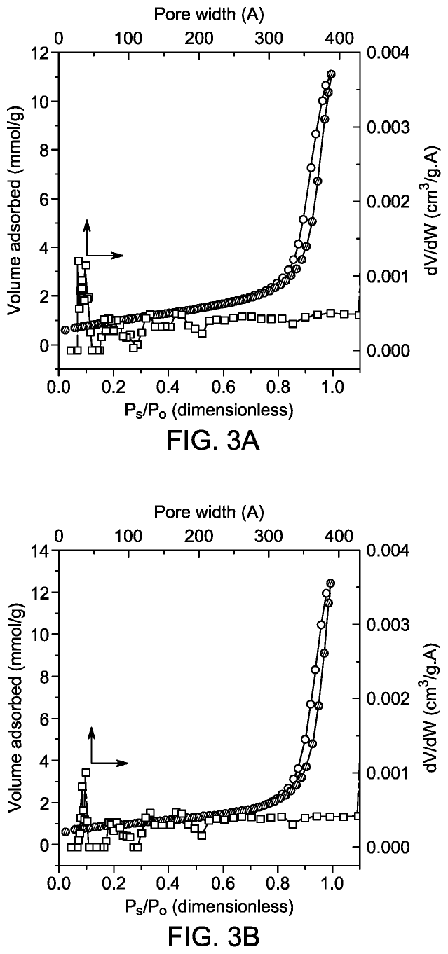

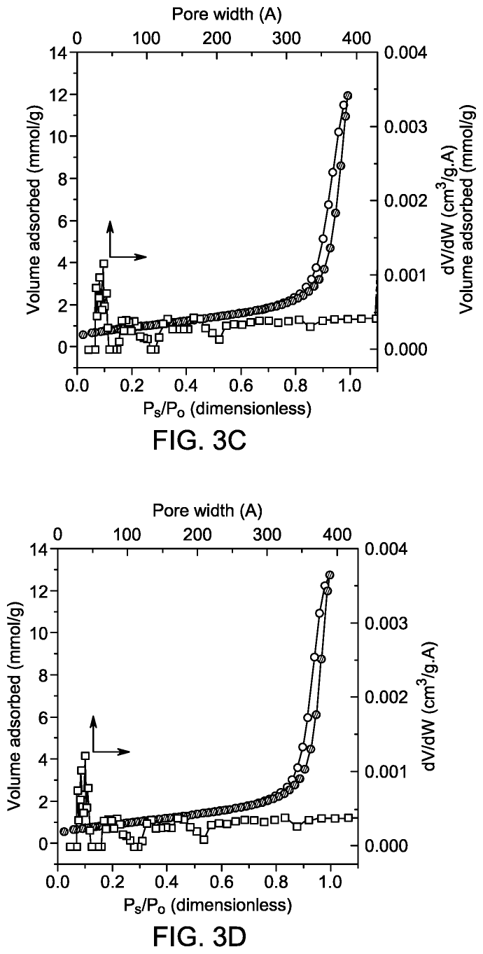

Referring to FIGS. 3A to 3D, N₂ adsorption-desorption isotherms of the active catalyst compositions. In some embodiments, the N₂-TPR was conducted on a Micromeritics ASAP 2020 sorption analyzer. In some embodiments, the active catalyst composition of the present disclosure has a total pore volume in a range of 0.1 to 1 cm²/g, preferably 0.1 to 0.8 cm²/g, preferably 0.2 to 0.6 cm²/g, and more preferably to 0.3 to 0.5 cm²/g, as depicted in FIGS. 3A to 3D. Other ranges are also possible.

As used herein, the term “temperature program desorption using ammonia,” or “NH₃-TPD” generally refers to a technique used to study the surface basicity of a solid material, such as an active catalyst. In some embodiments, the active catalyst is first heated in an inert gas, such as nitrogen, to remove any adsorbed species and to stabilize the surface. In some embodiments, the active catalyst is then cooled down and exposed to a stream of ammonia gas, which is adsorbed onto the surface of the active catalyst. The amount of ammonia adsorbed is proportional to the surface basicity of the active catalyst. In some embodiments, the active catalyst is then heated at a constant rate while the amount of ammonia desorbed is monitored as a function of temperature. In some further embodiments, as the temperature increases, the adsorbed ammonia begins to desorb from the surface of the active catalyst. In some preferred embodiments, the desorption of ammonia may be exothermic, and the heat generated by the desorption process is monitored using a thermal conductivity detector.

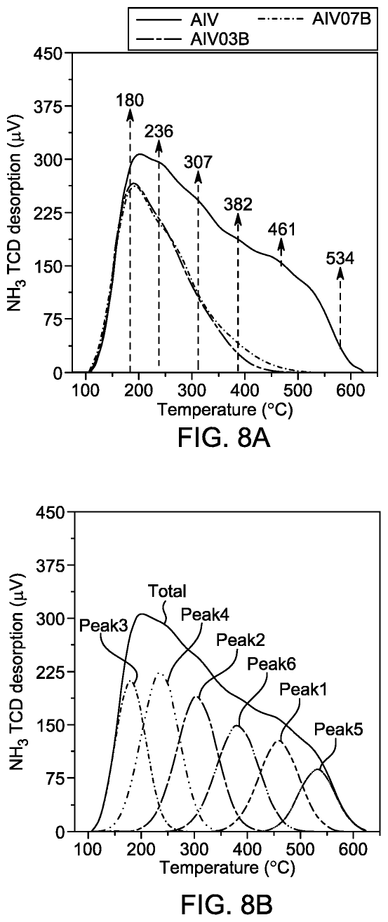

Referring to FIG. 8A, NH₃ temperature programmed desorption (NH₃-TPD) of the active catalyst compositions. In some embodiments, the NH₃-TPD was conducted on a BELCAT II Chemisorption analyzer. In some embodiments, NH₃-TPD of the active catalyst composition are about 0.01 to 0.5 mmol/g, preferably 0.05 to 0.4 mmol/g, preferably 0.1 to 0.3 mmol/g, or even more preferably about 0.2 mmol/g, as depicted in FIG. 8A. Other ranges are also possible.

Referring to FIG. 2B, a schematic flow diagram of the method 250 for producing propylene via oxidative dehydrogenation (ODH) of propane is illustrated. The order in which the method 250 is described is not intended to be construed as a limitation, and any number of the described method steps may be combined in any order to implement the method 250. Additionally, individual steps may be removed or skipped from the method 250 without departing from the spirit and scope of the present disclosure.

At step 252, the method 250 includes introducing a feed gas stream containing CO₂ and propane into a reactor containing the active catalyst composition. The volume ratio of CO₂ to propane in the feed gas stream is in a range of 1:10 to 10:1, preferably 1:1 to 5:1, and more preferably 1:1. The feed is a mixture of CO₂ and propane. Optionally, the feed may include other gases such as O₂, water vapor, nitrogen, or combinations thereof. The feed may also include promoters, such as carbon monoxide, nitrous oxide, N₂O, H₂O₂, O₃, and combinations thereof, to increase efficacy. The CO₂ and propane, optionally with other gases, are introduced into the reactor, as a mixture, through one or more inlets. In some embodiments, the reactor is at least one selected from the group consisting of a fixed-bed reactor, a trickle-bed reactor, a moving bed reactor, a rotating bed reactor, a fluidized bed reactor, and a slurry reactor. In an embodiment, the reactor is a fixed-bed reactor in the form of a vertical cylindrical reactor which includes a top portion, a vertically oriented cylindrical body portion, a bottom portion, a housing. In some embodiments, the housing has an open top, and open bottom supportably maintained with the vertically oriented cylindrical body portion. The active catalyst composition is supportably retained within the housing, permitting fluid flow therethrough. In some embodiments, the vertical cylindrical reactor further includes at least one propeller agitator disposed in the bottom portion of the reactor. In some embodiments, the main function of the propeller agitator is homogenization, dispersion, and suspension of low-viscosity products. In some embodiments, the bottom portion is cone-shaped or pyramidal. In an embodiment, the bottom portion may have a cylindrical, cubical, cuboidal, or rhombic shape. In some preferred embodiments, a plurality of recirculation tubes fluidly connects the bottom portion of the vertical cylindrical reactor with the vertically oriented cylindrical body portion of the vertical cylindrical reactor. In an embodiment, the fixed-bed reactor may be made up of a material such as stainless-steel, iron, aluminum, copper, lead, iron, zirconium, or another alloy.

At step 254, the method 250 includes passing the feed gas stream through the reactor in the presence of the active catalyst composition at a temperature of 300 to 900° C. to convert at least a portion of propane to propylene and produce a propylene-containing gas stream leaving the reactor. The pressure is maintained between 0.01 atm to 20 atmospheres. The conversion may be facilitated using UV, visible, or infrared light to promote the dehydrogenation reaction. The active catalyst composition converts at least a portion of propane to propylene. In some embodiments, the active catalyst composition converts at least 80% of propane to propylene based on the initial weight of the propane in the feed gas stream, preferably at least 90%, or even more preferably at least 99% based on the initial weight of the propane in the feed gas stream. The ODH reaction also results in a propylene-containing gas stream. The propylene-containing gas stream includes methane, ethane, ethylene, propane, carbon monoxide, carbon dioxide, a hydrocarbon containing C₄-C₉ such as butane, butene, pentane, and pentene; and aromatics such as benzene, naphthalene, anthracene, or their isomers, or mixtures of these substance. In some embodiments, the C4-C9 hydrocarbon contains butane, butene, butyne, pentane, pentene, pentyne, hexane, hexene, hexyne, cyclohexane, cyclohexene, heptane, heptene, heptyne, octane, octene, octyne, nonane, nonene, nonyne, or their isomers, or mixtures of these substance.

In some embodiments, the propylene is present in the propylene-containing gas stream at a concentration of 30 to 70 wt. %, more preferably 35 to 65 wt. %, preferably 40 to 60 wt. %, preferably 45 to 55 wt. %, each wt. % based on a total weight of the converted propane. In some embodiments, the ethylene is present in the propylene-containing gas stream at a concentration of 5 to 30 wt. %, more preferably 10 to 25 wt. %, or even more preferably 15 to 20 wt. %, each wt. % based on a total weight of the converted propane. In some embodiments, the ethane is present in the propylene-containing gas stream at a concentration of 0.001 to 5 wt. %, more preferably 0.01 to 3 wt. %, or even more preferably 0.1 to 2 wt. %, each wt. % based on a total weight of the converted propane. In some embodiments, the methane is present in the propylene-containing gas stream at a concentration of 1 to 20 wt. %, more preferably 5 to 15 wt. %, or even more preferably about 10 wt. %, each wt. % based on a total weight of the converted propane. Other ranges are also possible.

At step 256, the method 250 separating the propylene from the propylene-containing gas stream. The unreacted propane (exiting from the propylene-containing gas stream) may be fed back into the reactor through the inlet for the ODH process. The method of the present disclosure yields up to 50% of propylene based on the propane conversion, preferably up to 40%, or even more preferably up to 30% of propylene based on the propane conversion. About, 5 wt. % VOₓ/γ-Al₂O₃ and 5 wt. % BOₓ/γ-Al₂O₃ exhibited propane conversions of about 56 and 27%, along with propylene selectivity of about 35 and 57% at 625° C., respectively. The CO selectivity was about 42 and 11%, respectively. Tuning the active phase via controlled stabilization of BOX species regulated crucial properties of the 5 wt. % VOₓ/γ-Al₂O₃: (i) endowed suitable site diversity, (ii) balanced acidity, (iii) modulated the coordination environment of VOₓ species, and (iv) regulated surface reducibility. VOₓ—BOₓ/γ-Al₂O₃ composed of 7 wt. % boron has a propane conversion and propylene yield of 61 and 34%, respectively, over 12 h time on stream (TOS).

### EXAMPLES

The following examples demonstrate the catalytic composition, as described herein. The examples are provided solely for illustration and are not to be construed as limitations of the present disclosure, as many variations thereof are possible without departing from the spirit and scope of the present disclosure.

### Example 1: Methodology

A series of supported catalysts were synthesized via solvent- and template-free protocols. Before loading the active metal, an appropriate amount of commercial γ-alumina was hydrated in a beaker using distilled water. The content was constantly stirred with a magnetic stirrer for 3 h at room temperature. Next, the mixture was gradually heated at the rate of 3° C./min to a temperature of 120° C., and it was maintained until most of the water dried up. Finally, the content was dried in an oven at 120° C. for 2 h.

### Example 2: Synthesis of VOₓ/Al₂O₃(A1V)

3 g of support (dry weight equivalent) was mixed with 5 wt. % vanadium using a vanadyl acetyl acetate precursor. The mixture was thoroughly ground for 10 minutes until the precursor was homogeneously dispersed. Subsequently, around 3 g ethanol was added to enhance the dispersion, and the content was ground for 5 minutes. The paste was dried in an oven at 120° C. for 2 hours and later calcined via two-step calcination. The calcination involves ramped heating to 300° C. at 5° C./min and maintained for 2 h, then heated to 675° C. at the rate of 10° C./min and maintained for 6 h.

### Example 3: Synthesis of BOₓ(x)/Al₂O₃(A1B)

3 g of support (dry weight equivalent) was mixed in a beaker with 5 wt. % boron derived from boric acid (H₂BO₃) precursor. The mixture was thoroughly ground for 15 minutes until the precursor was homogeneously dispersed. The content was transferred to a furnace and calcined via a two-step calcination protocol. The calcination involves ramped heating to 300° C. at 5° C./min and maintained for 2 hours, then later heated to 650° C. at the rate of 10° C./min and maintained for 6 hours.

### Example 4: Synthesis of VOx-BOₓ(x)/Al₂O₃

4 g of alumina support (dry weight equivalent) was mixed with a target amount (3 or 7 wt. %) of boric acid precursor. The samples mixed with 3 wt. % of boric acid precursor are labeled AlV03B, while those mixed with 7 wt. % of boric acid precursor are labeled AlV07B. The same grinding protocol as above in Examples 1 to 3 was followed. The mixtures were then calcined at 400° C. at 5° C./min for 2 h. The calcined samples were removed from the furnace and mixed with 5 wt. % vanadium using a vanadyl acetyl acetate precursor. The mixtures were thoroughly ground for 10 min until the precursor was homogeneously dispersed. Subsequently, around 3 g ethanol was added to each mixture to enhance the dispersion, and the contents were ground for 5 min. The pastes were dried in an oven at 120° C. for 2 hours and later calcined via two-step calcination. The calcination involves ramped heating to 300° C. at 5° C./min and maintained for 2 hours, then later heated to 675° C. at the rate of 10° C./min and maintained for 6 hours.

The prepared samples A1V, A1B, AlV03B, and AlV07B were further characterized by various analytical techniques to probe the properties of fresh and spent catalysts to establish strong property-activity relationships.

### Example 5: UV-Vis DRS

UV-Vis DRS were acquired for the fresh samples on UV-VIS & UV-VIS-NIR Systems—Carry 5000 (manufactured by Agilent technologies, 5301 Stevens Creek Blvd Santa Clara, CA 95051. United States) in the range 200-800 nm in powder form. No standards or references, such as MgO or BaSO₄, were utilized. This enables probing of the coordination environment of the vanadium species over the supports.

### Example 6: NH₃-TPD

The surface acid-base properties of the catalysts were probed using NH₃-TPD on a BELCAT II Chemisorption analyzer (manufactured by Microtrac Inc, 215 Keystone Dr, Montgomeryville, Pennsylvania, 18936, United States). About 0.1 g of the catalyst sample was pre-heated at 500° C. for 60 minutes at a heating rate of 10° C./min. Next, the samples were cooled to 100° C. before ammonia adsorption for 30 minutes at 30 mL/min. Subsequently, the samples are flushed with helium and heated to 600° C. at 10° C./min. Finally, the signals were acquired using a thermal conductivity detector (TCD).

### Example 7: N₂ Adsorption Isotherms

N₂ adsorption analysis was conducted on a Micromeritics ASAP 2020 sorption analyzer (manufactured by Micromeritics Corporate Headquarters, 4356 Communications Dr. Norcross, GA 30093-2901, U.S.A) to probe the textural properties of the catalysts. About 100 mg of catalyst samples were degassed in a vacuum at 300° C. for 6 hours before the measurement in a liquid N₂ bath at −195.86° C. The BET method was utilized to quantify the specific surface areas, while the T-plot method was used to measure the external surface area and the mesoporous-microporous volumes of the catalysts. The pore distributions were measured via the NLDFT method.

### Example 8: Raman and Thermogravimetric Analysis (TGA)

Raman spectra of both fresh and spent catalysts were obtained using a Raman spectrometer at ambient temperature. The excitation source was a wavelength of 532 nm and at 25 mW. This enables the probing of the surface disorder on the catalysts via the D and G bands (ID/IG) ratio. The TGA of the spent samples was carried out to corroborate the Raman spectra results obtained regarding the extent of coke formation on the catalyst surface.

### Example 9: H₂ Reducibility

The surface reducibility of the fresh catalysts was probed via H₂ temperature programmed reduction (H₂-TPR) on Micromeritics AutoChem-II 2920 unit equipped with a TCD detector (manufactured by Micromeritics Corporate Headquarters, 4356 Communications Dr. Norcross, GA 30093-2901, U.S.A). About 100 mg of catalyst is pre-heated in a quartz tube to 300° C. under Ar flow regulated at 50 mL/min, and the temperature was maintained for 3 h. Before the temperature reduction analysis, the sample is cooled to room temperature. Subsequently, a mixture of Ar and 10% by volume H₂ was flown at the rate of 50 mL/min while linearly heating the sample at the rate of 10° C./min up to 850° C. The signal is recorded with calibrated TCD detector.

### Example 10: Catalytic Performance

Prior to the catalytic testing, the catalyst samples were pelletized, crushed, and sieved with a 330-345 mesh. In a defined run, 0.1 g of catalyst particles are loaded into a quartz reactor without diluting with inert materials such as SiC. Quartz wool was utilized to position the catalyst bed appropriately inside the reactor. The reactor model is a PID Microactivity-Effi reactor (manufactured by Micromeritics Corporate Headquarters, 4356 Communications Dr. Norcross, GA 30093-2901, U.S.A); a fully-automated reactor connected to an online 7820A Agilent gas chromatograph equipped with HP-Plot-Q capillary column along with both TCD and FID detectors (manufactured by Agilent technologies, 5301 Stevens Creek Blvd Santa Clara, CA 95051. United States). All gaseous products were analyzed accordingly, and the line connecting the reactors with the GC is constantly maintained at 120° C. to ensure that the water produced during the CO₂—ODHP does not condense inside. The flow into the reactor is regulated using Bronkhorst mass flow meters (manufactured by Bronkhorst Instruments GmbH, Am Ziegelwerk 1, 85391 Allershausen, Germany). The loaded catalyst is usually pre-treated with air at a flow rate of 30 mL/min while the bed is ramped to 675° C. at 20° C./min. Subsequently, the reactor temperature is cooled to the desired temperature for reaction, and the gas is switched to helium and maintained for 30 min to flush the catalyst and prepare it for reaction. Later, the reactor is isolated by activating the bypass valve, and the feed (mixture of propane and CO₂ at a ratio of 1:1) is flown continuously. When the flow stabilizes (after around 10 min), the reactor is opened while the GC valves are activated after 2 min. The GC records the full FID and TCD chromatogram every 10 minutes. The GC was carefully calibrated with a standard gas mixture previously. Notably, the feed conversion, selectivity, and yield to a particular product are defined by equations (3) and (4), respectively.

$$Propane conversion , {X}_{{C}_{3} {H}_{8}} = \frac{{( {n}_{{C}_{3} {H}_{8}} )}_{in} - {( {n}_{{C}_{3} {H}_{8}} )}_{out}}{{( {n}_{{C}_{3} {H}_{8}} )}_{in}} \times 100 \% \qquad ( 1 )$$

$${CO}_{2} conversion , {X}_{{C}_{3} {H}_{8}} = \frac{{( {CO}_{2} )}_{in} - {( {CO}_{2} )}_{out}}{{( {CO}_{2} )}_{in}} \times 100 \% \qquad ( 2 )$$

$$Hydrocarbons selectivity , {S}_{i} = \frac{{z}_{j} {n}_{j}}{3 ( {n}_{{C}_{3} {H}_{{8}_{in}}} - {n}_{{C}_{3} {H}_{{8}_{out}}} )} \times 100 \% \qquad ( 3 )$$

$$CO selectivity , {S}_{i} = \frac{0.5 * ( {n}_{CO} - {n}_{{H}_{2} O} )}{3 ( {n}_{{C}_{3} {H}_{{8}_{in}}} - {n}_{{C}_{3} {H}_{{8}_{out}}} )} \times 100 \% \qquad ( 4 )$$

$$Product yield , {Y}_{i} = ( {X}_{{C}_{3} {H}_{8}} * {S}_{i} ) \times 100 \% \qquad ( 5 )$$

where (n_{C}_{₃}_{H}_{₈})ᵢₙ and (n_{C}_{₃}_{H}_{₈})ₒᵤₜ denote the inlet and outlet moles of the propane feed, while zⱼ and nⱼ denotes the number of carbons and moles of particular product species j.

### Example 11: Catalyst Characterization

TABLE 1
Textural, acidity, and surface reducibility properties for the catalysts

| | N2 adsorption/desorption | | | | NH₃-TPD | H₂-TPR |
|---|---|---|---|---|---|---|
| | | | Total pore | Mesopore | Total | Surface |
| | S_{BET} | Smicro | volume | volume | acidity | reducibility |
| Catalysts | (m²/g · cat) | (m²/g · cat) | (cm²/g · cat) | (cm²/g · cat) | (mmol/g) | (mmol/g H₂) |
| AlB | 74.9 | 12.4 | 0.432 | 0.426 | — | |
| AlV | 80.5 | 5.8 | 0.386 | 0.383 | 0.250 | 0.317 |
| AlV03B | 76.6 | — | 0.413 | — | 0.108 | 0.290 |
| AIV07B | 76.0 | — | 0.443 | — | 0.113 | 0.185 |

### Example 12: N₂-Adsorption/Desorption Analysis

The textural properties of the various catalysts investigated were probed via N₂-adsorption/desorption analysis. FIG. 3A-D depict the results along with NLDFT-derived pore size distributions. All the catalysts showed similar adsorption phenomena characterized by weak adsorbent-adsorbate interactions coupled with pore filling, as evident from their Type 5 isotherms (elongated S-type) (FIGS. 3A-3D). The pore structures are likely 2D cylindrical as defined by the H1 hysteresis loops that characterized mesoporous materials. Nonetheless, the microporous features of the catalysts are uniquely depicted by N₂ uptake occurring at lower P/Po, and uptakes at P/Po=0.2 and 0.9 characterize intra-particle and inter-particle pore features, respectively. However, the specific surface areas of all the catalysts are low, less than 100 μm²/g·cat, as shown in Table 1.

### Example 13: FTIR

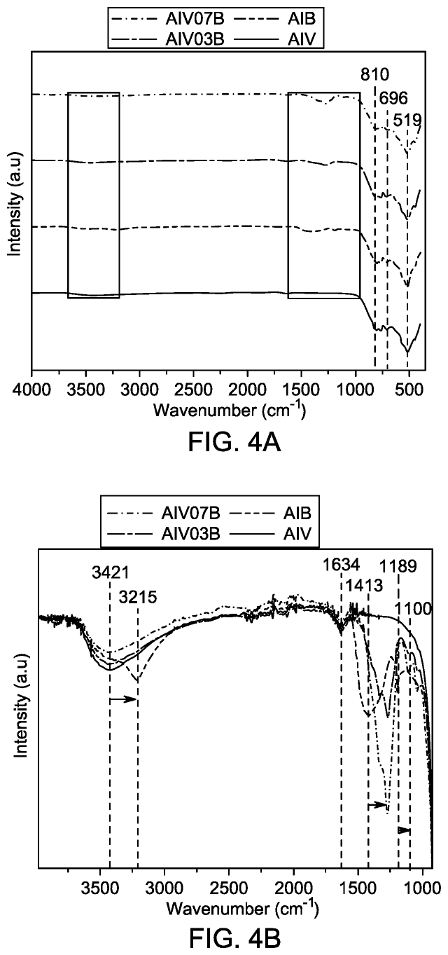

FIG. 4A depicts the FTIR spectra of various as-synthesized catalysts in the range 400-4000 cm⁻¹. All the catalysts exhibited similar spectra with adsorption bands at 810, 696, and 519 cm⁻¹ indicating vibration of the Al—O bonds. FIG. 4B shows the spectra between 1000-4000 cm⁻¹, highlighting the changes from incorporating the boron species (BOₓ). All the catalysts exhibited a band with wavenumber 3421 cm⁻¹ attributed to —OH bonds; however, the AlB sample showed a pronounced extra band at 3215 cm⁻¹.

### Example 14: UV-Vis DRS

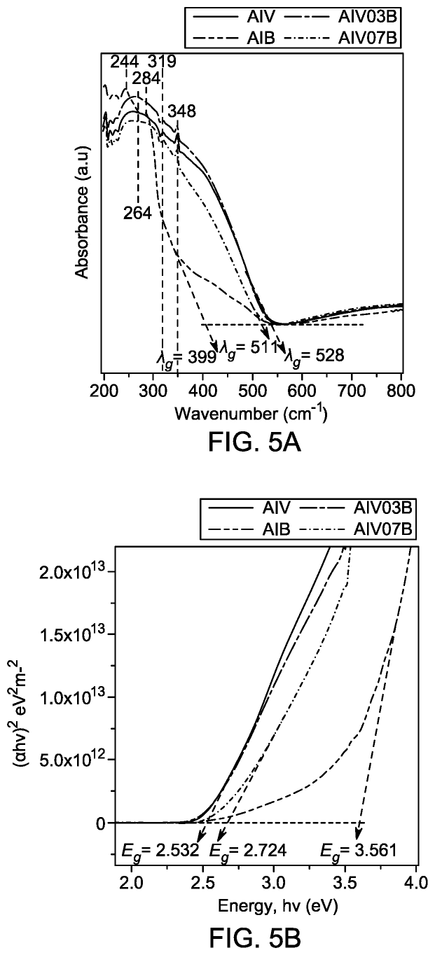

The local coordination environment of the VOx species was investigated by UV-vis DRS. The technique is adequate to probe the changes in the coordination environment, but quantitative measurements are only guaranteed at a low loading of vanadium oxide due to the complexity associated with possible bands overlapping. FIG. 5A depicts the absorbance versus wavelength (200-800 nm). The bands located at 264, 284, 319, and 348 nm are attributed to ligand-to-metal charge transfer (LMCT, O²⁻→V⁵⁺) in isolated and less polymerized VOx species. Such transfers occur due to the availability of low-lying empty orbitals in the vanadium metal species. Comparing the AlV and AlV07B spectra reveals a significant broadening and shifting of the electronic absorption bands to higher wavelengths in the latter, indicating that the VOx species in AlV are more polymerized. In other words, polymerized samples have more pronounced bands between 348-500 nm. Moreover, the plot of (αhν)² versus incident photo energy for band gap evaluation is depicted in FIG. 5B corroborated this finding. This is because the AlV07B catalyst possesses significantly higher band gap energy (2.724 eV) than AlV (2.532 eV). Incorporating BOx as another distinct active species partly isolated the VOx species and made them less polymerized.

### Example 15: Raman Spectroscopy

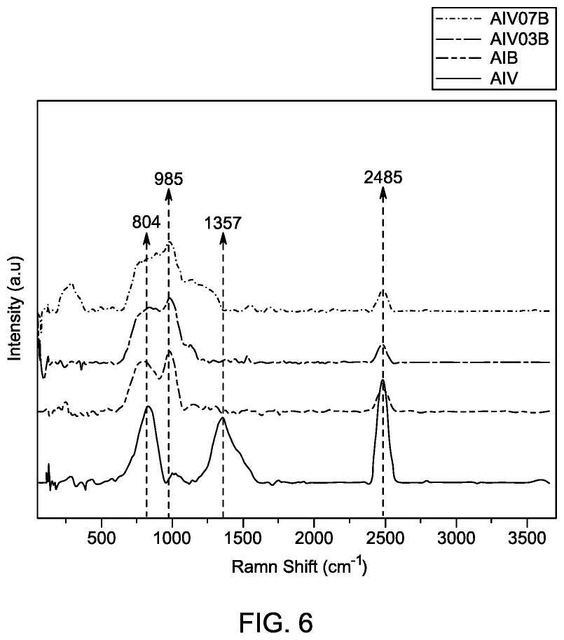

The evolution of surface-active species was also probed by Raman spectroscopy. It is a powerful technique that can provide great detail regarding the nature of active sites available on the catalyst surface. FIG. 6 depicts the spectra of all the as-synthesized catalysts. The incorporation of BOₓ active species caused the obvious extinction of the electronic absorption band located at 1357 nm in the AlV catalyst. This shows that the boron species interacted chemically with the sites located there. Similarly, the band located at 2485 nm exhibited a severe decline in intensity in the modified AlV03 and AlV07B catalysts, which may be attributed to unknown organic impurities in the samples.

### Example 16: Surface Reducibility

FIG. 7 depicts the results of the H₂-TPR analysis of the catalysts. From FIG. 7A, the profiles reveal significant variation in the surface reducibility of the active phase of the catalysts due to the incorporation of BOx species. As shown in Table 1, the total surface reducibility of the AlV03B and AlV07B were 0.290 and 0.185 mmol/g H₂, respectively, whereas AlV exhibited a value of around 0.317 mmol/g H₂. In addition, the construction of active site diversity via integrating the BOx sites significantly narrowed the temperature range for the reduction and simultaneously shifted it to a lower temperature region. From FIG. 7B, the AlV, AlVO3B, and AlV07B showed their reduction temperature in the range 397-849, 322-727, and 356-781° C., respectively, as can be observed from the peaks 1-6. The observation regarding the surface reducibility of the catalysts can be anticipated to result in a significant difference in the catalytic behavior of the catalysts.

### Example 17: NH₃-TPD

The surface nature and concentration of available acid sites on the catalysts were investigated via NH₃-TPD. FIG. 8A and FIG. 8B depict the NH₃-TPD profiles, and it is evident that the introduction of active site diversity via integrating the BOₓ sites modulated the surface acidity of the catalysts. The desorption peaks of AlV03B and AlV07B appeared within a narrower range (110-495° C.) than that of the AlV catalyst (110-625° C.). This indicates that the active site diversity moderates the strength of acid sites. Table 1 shows the total concentration of acid sites for the AlV, AlV03B, and AlV07B is 0.250, 0.101, and 0.113 mmol/g, respectively. This indicates that not only was the strength of acid sites regulated, but the concentrations of the sites were also moderated significantly. The trend appears similar to what was observed in the H₂-TPR analysis. Overall, the modulation of the surface acidity and other factors impacted the propane CO₂—ODH performance of the catalysts.

### Example 18: Catalytic Performance

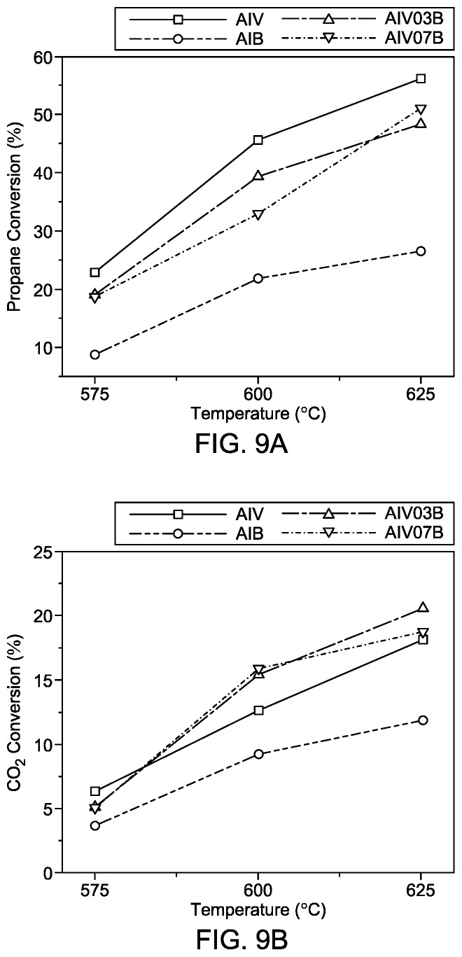

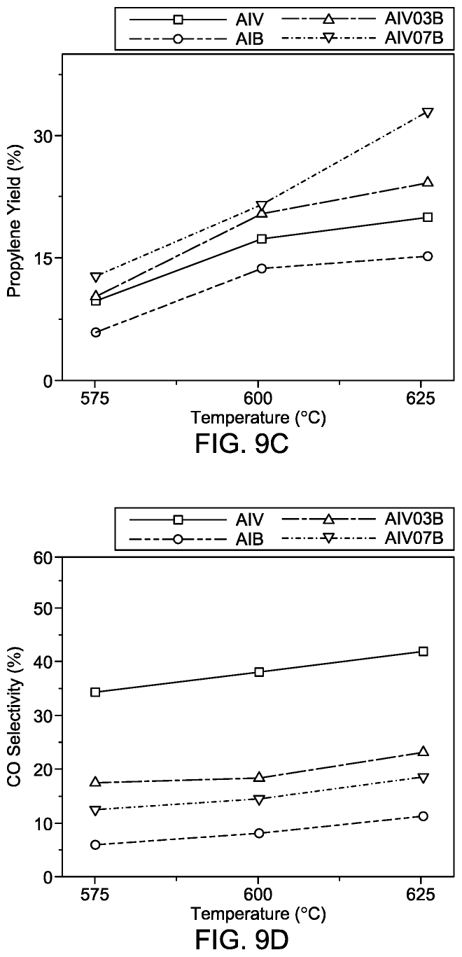

FIG. 9 shows the results of the catalytic performance evaluation of the various catalysts. The results were acquired after 30 h TOS. The propane conversions for all the catalyst increase significantly with increased reaction temperature as depicted in FIG. 9A. Incorporating boron into the active phase lowered the observable activity, as evident from the highest propane conversion of the unmodified AlV catalyst. At 625° C., the AlV exhibited nearly double the propane conversion of the AlB catalyst (56%). A similar trend was observed for CO₂ conversions (FIG. 9B). However, the AlV03 and AlV07B catalysts indicated comparable activity to the AlV samples, especially at higher temperatures.

The construction of active site diversity revealed significant improvement in overall propylene yield, as evident from the superior performance of the AlV03 and AlV07B. The improvement is related to lowering the acid site concentrations and regulating the surface reducibility, as evident from the NH3-TPD and H2-TPR analysis (depicted in Table 1). A high concentration of acid sites, especially those of high strength, usually inhibits the desorption of generated olefin products and promotes the chances of their cracking and possible overoxidation. From another perspective, many investigations found that boron-based catalysts usually have excellent olefin selectivity. Their low-temperature catalytic performance is unmatched [W. D. Lu, D. Wang, Z. Zhao, W. Song, W. C. Li, A. H. Lu, ACS Catal. 9 (2019) 8263-8270, which is incorpotated herein by reference in its entirety]. Thus, the superior overall propylene yields exhibited by the AlV03 and AlV07B (FIG. 9C) coupled with their lowest CO selectivity (FIG. 9D) could also be firmly attributed to the presence of suitable active site diversity. This is because the controlled incorporation of the BOx active species modulated the surface coordination state of the VOx species, as evidenced by the significant increase in the band gap energies of the AlV03 and AlV07B catalysts (FIG. 5). The structure-performance relationship of series of vanadium-based catalysts revealed that surface enrichment of VOx species positively correlates with decreased band gap energies [P. Bai, Z. Ma, T. Li, Y. Tian, Z. Zhang, Z. Zhong, W. Xing, P. Wu, X. Liu, Z. Yan, ACS Appl. Mater. Interfaces 8 (2016) 25979-25990, which is incorporated herein by reference in its entirety]. Therefore, this indicates that over AlV03 and AlV07B surfaces, polymerized VOx species are adequately separated compared to the AlV, as evidenced by their higher band gap energies. More polymerized surface VOₓ species (having V—O—V—O chains) are usually highly active but less selective in oxidative dehydrogenation due to their tendencies for undesired oxygen insertion reactions [L. Liu, X. Han, J. Zhou, M. Zhang, M. Wu, K. Fang, J. Porous Mater. 25 (2018) 955-963, which is incorporated herein by reference in its entirety]. Unlike when other elements such as phosphorous are utilized to achieve desired isolation of VOₓ species [Y. Gu, H. Liu, M. Yang, Z. Ma, L. Zhao, W. Xing, P. Wu, X. Liu, S. Mintova, P. Bai, Z. Yan, Appl. Catal. B Environ. 274 (2020), which is incorporated herein by reference in its entirety], the controlled incorporation of BOx species serves the dual purpose of regulating the local coordination environment and constructing additional unique sites to augment the selective oxidative role synergistically.

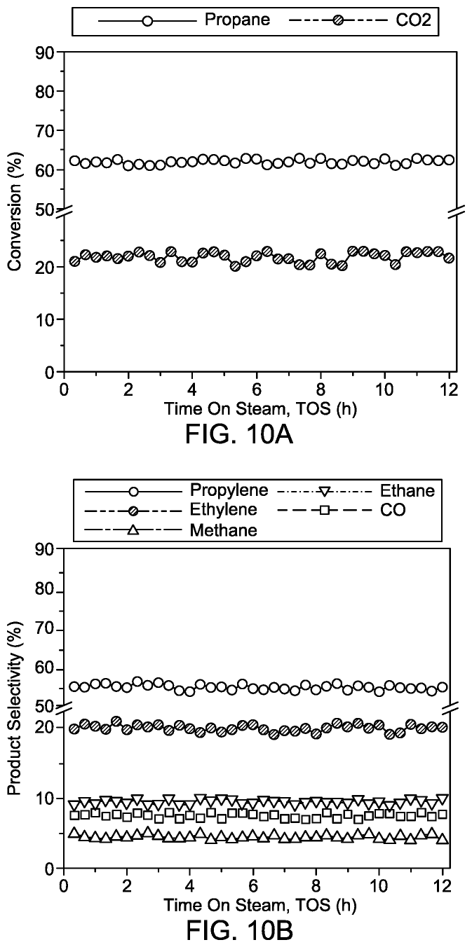

Another requirement for prospective catalysts in a particular application is the exhibition of stable catalytic performance over a long TOS. FIG. 10A depicts the catalytic stability of the AlV07B catalyst, revealing that the catalyst was highly stable. Moreover, both propane and CO₂ conversion rates showed no noticeable decline over 12 h TOS (FIG. 10B). Overall, the catalyst realized a remarkable 34% yield to desired propylene product couple with less than 6% yield to unwanted CO, showing that the suitable active site diversity of the AlV07B catalyst endowed it robust catalytic features that dynamically maintained the synergy among the distinct sites.

### Example 19: TGA Analysis

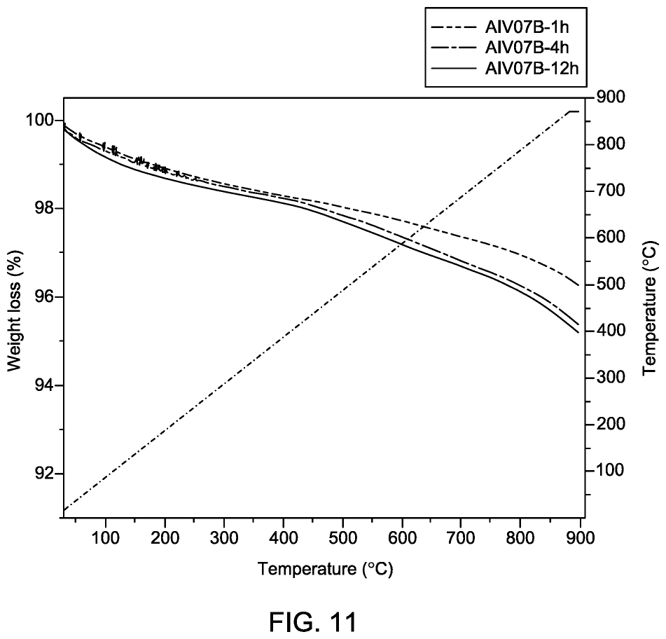

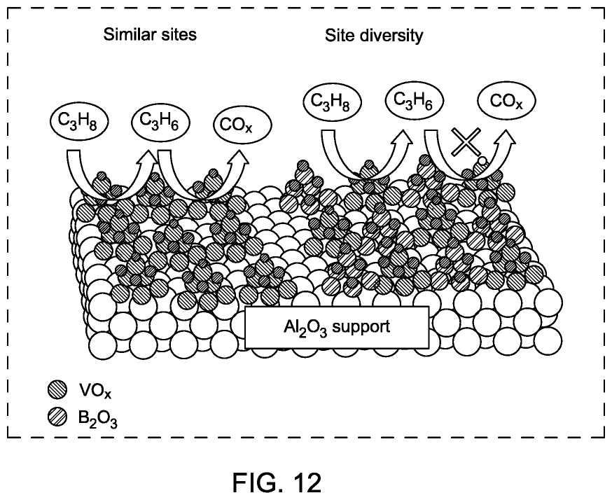

The spent AlVO7B catalyst from the stability test was subjected to TGA to assess the extent of possible coke formation. From FIG. 11, it shows that little amount of carbon deposition formed on the catalyst surface, which mainly occurred in the first 4 h of the TOS. FIG. 12 shows a schematic illustration of promoting catalytic performance in CO₂-mediated oxidative dehydrogenation by constructing suitable active site diversity. Integrating boron active species (BOx) with moderate adsorption ability regulates the overall acidity of the catalyst surface and enable enables the desorption of generated propylene and ethylene products before being cracked and overoxidized.

A facile template-free strategy for developing promising heterogeneous catalysts for CO₂—ODH of propane is demonstrated. The synthesis protocol involves two active species—VOx and BOx—to harness their catalytic abilities while effectively circumventing their individual drawbacks via triggered synergistic interaction among the stabilized active sites. Incorporating boron species positively promoted catalytic performance, with AlV07B composed of 7 wt. % boron exhibiting a high propylene yield of 34% coupled with less than 10% CO selectivity at 650° C., over 12 h TOS without observable deactivation, despite TGA analysis revealing a small amount of coke formation, especially in the early first 4 h of reaction.

Numerous modifications and variations of the present disclosure are possible in light of the above teachings. It is therefore to be understood that within the scope of the appended claims, the invention may be practiced otherwise than as specifically described herein.

1: A method of making an active catalyst composition containing a first catalyst and a second catalyst, comprising:
mixing an alumina support having an average particle size of 5 to 100 micrometers (μm) with first catalyst precursor particles and grinding thereby at least partially embedding the first catalyst precursor particles onto surfaces of the alumina support to form a first composite precursor;
wherein a weight ratio of the alumina support to the first catalyst precursor particles is in a range of 100:1 to 5:1;
mixing the first composite precursor and a first solvent to form a first mixture;
grinding the first mixture and drying at a temperature of 100 to 150° C.; and
calcining the first mixture after the drying at a first temperature of at least 200° C. and a second temperature of at least 550° C. thereby allowing the first catalyst precursor particles embedded onto the surfaces of the alumina support to decompose in situ to generate first catalyst particles embedded onto the surfaces of the alumina support and form the first catalyst;
wherein the first catalyst particles have an average particle size in a range of 20 to 200 nanometers (nm);
wherein the first catalyst particles comprise vanadium oxide (VOₓ) particles, and boron oxide (B₂O₃) particles;
wherein 0<x<3; and
mixing the first catalyst with the second catalyst to form the active catalyst composition.

2: The method of claim 1, wherein the alumina support is at least one selected from the group consisting of alpha-alumina, delta-alumina, theta-alumina, and gamma-alumina.

3: The method of claim 1, wherein the first catalyst precursor particles comprise a vanadium compound selected from the group consisting of vanadium acetylacetate, vanadium acetylacetonate, ammonium vanadate, vanadyl oxalate, vanadium pentoxide, vanadium monoethanolamine, vanadium chloride, vanadium trichloride oxide, vanadyl sulfate, vanadium antimonate, antimony vanadate, vanadium oxyacetylacetonate, vanadium oxyacetate, vanadium oxyhalide, and vanadium oxytriisopropoxide.

4: The method of claim 1, wherein the first catalyst precursor particles comprise a boron compound selected from the group consisting of boric acid, boron nitride, borax, boron halide, and borane halide.

5: The method of claim 1, wherein the vanadium oxide (VOₓ) particles comprise vanadium monoxide (VO), vanadium trioxide (V₂O₃), vanadium dioxide (VO₂), and vanadium pentoxide (V₂O₅).

6: The method of claim 1, further comprising:
forming the second catalyst by:
mixing the first catalyst with second catalyst precursor particles and grinding thereby at least partially embedding the second catalyst precursor particles onto the surfaces of the alumina support to form a second composite precursor;
wherein a weight ratio of the first catalyst to the second catalyst precursor particles is in a range of 100:1 to 9:1;
mixing the second composite precursor and a second solvent to form a second mixture; and
grinding the second mixture and drying at a temperature of 100 to 150° C.;
calcining the second mixture after the drying at a first temperature of at least 200° C. and a second temperature of at least 550° C. thereby allowing the second catalyst precursor particles to decompose in situ to generate second catalyst particles embedded onto the surfaces of the alumina support and form the second catalyst;
wherein the second catalyst particles have an average particle size in a range of 20 to 200 nm; and
wherein the second catalyst particles comprise vanadium oxide particles, and boron oxide particles.

7: The method of claim 6, wherein the second solvent is at least one selected from the group consisting of a ketone solvent, an ester solvent, an alcohol solvent, an amide solvent.

8: The method of claim 6, wherein the second composite precursor is present in the second mixture at a concentration of 40 to 80 wt. % based on a total weight of the second mixture.

9: The method of claim 1, wherein the active catalyst composition is at least one selected from the group consisting of an alumina supported vanadium oxide (VOx), an alumina supported boron oxide (B₂O₃), and an alumina supported VOx/B₂O₃; and
wherein 0<x<3.

10: The method of claim 1, wherein the active catalyst composition has a layered mesoporous structure.

11: The method of claim 1, wherein the active catalyst composition has a specific surface area in a range of 50 to 100 square meters per gram (m²/g).

12: The method of claim 1, wherein the active catalyst composition has a cumulative specific pore volume in a range of 0.2 to 0.7 cubic centimeters per gram (cm³/g).

13: The method of claim 1, wherein the active catalyst composition has an average pore diameter of 50 to 400 angstroms (Å).

14: The method of claim 1, wherein the active catalyst composition has a temperature-programmed desorption of ammonia (NH₃-TPD) of 0.01 to 0.5 millimoles per gram (mmol/g).

15: The method of claim 1, wherein the active catalyst composition has a hydrogen temperature-programmed reduction (H₂-TPR) of 0.01 to 0.7 mmol/g.

16: A method for producing propylene via oxidative dehydrogenation (ODH) of propane, comprising:
introducing a feed gas stream containing CO₂ and propane into a reactor containing the active catalyst composition prepared by the method of claim 1;
passing the feed gas stream through the reactor in the presence of the active catalyst composition at a temperature of 300 to 900° C. to convert at least a portion of the propane to propylene and produce a propylene-containing gas stream leaving the reactor; and
separating the propylene from the propylene-containing gas stream.

17: The method of claim 16, wherein a volume ratio of CO₂ to propane in the feed gas stream is in a range of 1:10 to 10:1.

18: The method of claim 16, wherein the propylene-containing gas stream further comprises methane, ethane, ethylene, propane, carbon monoxide, carbon dioxide, hydrocarbon containing C4-C5, and aromatics.

19: The method of claim 16, having a propane conversion of up to 80% based on an initial weight of the propane in the feed gas stream.

20: The method of claim 16, having a propylene yield of up to 50% based on the propane conversion according to equation Y₃=(X_{C}_{₃}_{H}_{₈}*S₃)×100%;
wherein X_{C}_{₃}_{H}_{₈} denotes the propane conversion to propylene; and
wherein S₃ denotes the propylene selectivity.
